(19) United States
(12) Patent Application Publication
(10) Pub. No.: US 2022/0242966 A1
(43) Pub. Date: Aug. 4, 2022

(54) CD63 AGONIST-RELATED METHODS AND COMPOSITIONS

(71) Applicant: AgonOx, Inc., Portalnd, OH (US)

(72) Inventors: Ryan Montler, Camas, WA (US); Lili Liu, Vancouver, WA (US); Grant Risdon, Seattle, WA (US)

(21) Appl. No.: 17/629,295

(22) PCT Filed: Jul. 23, 2020
(86) PCT No.: PCT/US2020/043264

§ 371 Date: Jan. 21, 2022

Related U.S. Application Data
(60) Provisional application No. 62/878,069, filed on Jul. 24, 2019.

(51) Int. Cl. C07K 16/28; A61P 35/00

(52) U.S. Cl. CPC C07K 16/2896; C07K 16/2878; A61K 2039/507; A61P 35/00; C07K 16/2809; C07K 16/2827

(57) ABSTRACT

Provided are methods of treating cell proliferative disorders. In some embodiments, the methods include administering to a subject having a cell proliferative disorder a therapeutically effective amount of a CD63 agonist, where the CD63 agonist is administered to the subject to enhance a T cell response to abnormally proliferating cells of the cell proliferative disorder. In certain embodiments, the methods include administering to a subject having a cell proliferative disorder a therapeutically effective amount of a CD63 agonist and a therapeutically effective amount of a T cell activator. Also provided are pharmaceutical compositions and kits that find use, e.g., in practicing the methods.

### CROSS-REFERENCE TO RELATED APPLICATIONS

This application claims the benefit of U.S. Provisional Patent Application No. 62/878,069, filed Jul. 24, 2019, which application is incorporated herein by reference in its entirety.

### SUMMARY

Provided are methods of treating cell proliferative disorders. In some embodiments, the methods include administering to a subject having a cell proliferative disorder a therapeutically effective amount of a CD63 agonist, where the CD63 agonist is administered to the subject to enhance a T cell response to abnormally proliferating cells of the cell proliferative disorder. In certain embodiments, the methods include administering to a subject having a cell proliferative disorder a therapeutically effective amount of a CD63 agonist and a therapeutically effective amount of a T cell activator. Also provided are pharmaceutical compositions and kits that find use, e.g., in practicing the methods.

### BRIEF DESCRIPTION OF THE FIGURES

FIG. 1: A graph showing co-stimulation of human T cells via CD63 and CD3 using a first anti-CD63 antibody agonist.

FIG. 2: A graph showing co-stimulation of human T cells via CD63 and CD3 using a second anti-CD63 antibody agonist.

FIG. 3: Flow cytometry data showing CD63 expression levels on T cells in human ovarian cancer.

FIG. 4: Flow cytometry data showing CD63 expression levels on tumor infiltrating lymphocytes (TILs) relative to blood in a human head and neck cancer patient.

FIG. 5: Data demonstrating the in vivo efficacy of a CD63 agonist in treating cancer.

### DETAILED DESCRIPTION

Before the methods, compositions and kits of the present disclosure are described in greater detail, it is to be understood that the methods, compositions and kits are not limited to particular embodiments described, as such may, of course, vary. It is also to be understood that the terminology used herein is for the purpose of describing particular embodiments only, and is not intended to be limiting, since the scope of the methods, compositions and kits will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods, compositions and kits. The upper and lower limits of these smaller ranges may independently be included in the smaller ranges and are also encompassed within the methods, compositions and kits, subject to any specifically excluded limit in the stated range. Where the stated range includes one or both of the limits, ranges excluding either or both of those included limits are also included in the methods, compositions and kits.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods, compositions and kits belong. Although any methods, compositions and kits similar or equivalent to those described herein can also be used in the practice or testing of the methods, compositions and kits, representative illustrative methods, compositions and kits are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods, compositions and kits are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods, compositions and kits, which are, for clarity, described in the context of separate embodiments, may also be provided in combination in a single embodiment. Conversely, various features of the methods, compositions and kits, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods, compositions and kits and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

Methods

Aspects of the present disclosure include methods of treating cell proliferative disorders. According to some embodiments, the methods include administering to a subject having a cell proliferative disorder a therapeutically effective amount of a CD63 agonist, where the CD63 agonist is administered to the subject to enhance a T cell response to abnormally proliferating cells of the cell proliferative disorder. The methods of the present disclosure are based in part on the inventors' surprising discovery that CD63 agonists enhance T cell responses to abnormally proliferating cells of a cell proliferative disorder (e.g., cancer), independent of whether the abnormally proliferating cells express or overexpress CD63. As such, in certain embodiments, the CD63 agonist is administered to the subject independent of the level of expression of CD63 on the abnormally proliferating cells of the cell proliferative disorder. According to some embodiments, the methods include administering to a subject having a cell proliferative disorder a therapeutically effective amount of a CD63 agonist, where at the time of the administering, abnormally proliferating cells of the cell proliferative disorder are not suspected of exhibiting overexpression of CD63. By “not suspected of exhibiting overexpression of CD63” is meant that CD63 is not suspected to be expressed at higher levels on the abnormally proliferating cells (e.g., cancer cells) compared to a second cell population (e.g., non-abnormally proliferating (e.g., non-cancer) cells of the same cell/tissue type as the abnormally proliferating cells). In certain embodiments, at the time of the administering, it has been determined that abnormally proliferating cells of the cell proliferative disorder do not overexpress CD63. According to some embodiments, the methods include making such a determination. In certain embodiments, the subject to whom the CD63 agonist is administered is receiving a T cell activation therapy. A non-limiting example of such a T cell activation therapy is an immune checkpoint inhibitor therapy, e.g., a therapy where the subject is receiving one or more inhibitors of PD-1, PD-L1, CTLA-4, LAG-3, TIM-3, TIGIT, VISTA, B7-H3, and/or the like. The methods of the present disclosure may further include administering the T cell activation therapy to the subject. Accordingly, aspects of the present disclosure include methods of treating cell proliferative disorders, the methods including administering to a subject having a cell proliferative disorder a therapeutically effective amount of a CD63 agonist and a therapeutically effective amount of a T cell activator. Details regarding embodiments of the methods of the present disclosure will be now be described.

The tetraspanins are integral membrane proteins expressed on cell surface and granular membranes of hematopoietic cells and are components of multimolecular complexes with specific integrins. The tetraspanin CD63 (also known as LAMP-3, melanoma-associated antigen ME491, TSPAN30, MLA1 and OMA81H) is a lysosomal membrane glycoprotein that translocates to the plasma membrane after platelet activation. CD63 (UniProtKB—P08962) functions as cell surface receptor for TIMP1 and plays a role in the activation of ITGB1 and integrin signaling, leading to the activation of AKT, FAK/PTK2 and MAP kinases. CD63 is expressed on activated platelets, monocytes and macrophages, and is weakly expressed on granulocytes, T cell and B cells. It is located on the basophilic granule membranes and on the plasma membranes of lymphocytes and granulocytes. CD63 is a member of the TM4 superfamily of leukocyte glycoproteins that includes CD9, CD37 and CD53, which contain four transmembrane regions. CD63 may play a role in phagocytic and intracellular lysosome-phagosome fusion events. CD63 deficiency is associated with Hermansky-Pudlak syndrome.

The CD63 agonist may be administered to any of a variety of subjects. In certain embodiments, the subject is a “mammal” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). According to some embodiments, the subject is a human. In certain embodiments, the subject is an animal model (e.g., a mouse model, a primate model, or the like) of a cellular proliferative disorder, e.g., cancer.

As summarized above, the subject has a cell proliferative disorder. By “cell proliferative disorder” is meant a disorder wherein unwanted cell proliferation of one or more subset(s) of cells in a multicellular organism occurs, resulting in harm, for example, pain or decreased life expectancy to the organism. Cell proliferative disorders include, but are not limited to, cancer, pre-cancer, benign tumors, blood vessel proliferative disorders (e.g., arthritis, restenosis, and the like), fibrotic disorders (e.g., hepatic cirrhosis, atherosclerosis, and the like), psoriasis, epidermic and dermoid cysts, lipomas, adenomas, capillary and cutaneous hemangiomas, lymphangiomas, nevi lesions, teratomas, nephromas, myofibromatosis, osteoplastic tumors, dysplastic masses, mesangial cell proliferative disorders, and the like.

In some embodiments, the subject has cancer. The subject methods may be employed for the treatment of a large variety of cancers by virtue of the enhanced anti-cancer T cell response achieved. In some embodiments, the subject has a cancer suspected of evading the immune system (e.g., effector T cells), e.g., by co-opting one or more immune checkpoint pathways. “Tumor”, as used herein, refers to all neoplastic cell growth and proliferation, whether malignant or benign, and all pre-cancerous and cancerous cells and tissues. The terms “cancer” and “cancerous” refer to or describe the physiological condition in mammals that is typically characterized by unregulated cell growth/proliferation. Examples of cancers that may be treated using the subject methods include, but are not limited to, carcinoma, lymphoma, blastoma, and sarcoma. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, prostate cancer, vulval cancer, thyroid cancer, hepatic carcinoma, various types of head and neck cancer, and the like. In certain aspects, the subject has a cancer selected from melanoma, Hodgkin lymphoma, renal cell carcinoma (RCC), bladder cancer, non-small cell lung cancer (NSCLC), and head and neck squamous cell carcinoma (HNSCC). In some embodiments, the subject has a cancer for which administration of a T cell activator (e.g., an immune checkpoint inhibitor (e.g., a CTLA-4 inhibitor, a PD-1 inhibitor, a PD-L1 inhibitor, a LAG-3 inhibitor, a TIM-3 inhibitor, an IDO inhibitor, a TIGIT inhibitor, a VISTA inhibitor, a B7/H3 inhibitor, and/or the like), an agonist of a T cell co-stimulatory receptor, an antagonist of a T cell inhibitory signal, and/or the like) to treat the cancer (alone or in combination with second anti-cancer agent) has been approved by the Food and Drug Administration (FDA) and/or the European Medicines Agency (EMA).

By “CD63 agonist” is meant an agent determined to have activity in agonizing CD63. An example assay for determining whether a CD63 binding agent is a CD63 agonist is one in which co-stimulation of CD4 T cells is assessed for a plate-bound CD63 agonist and the corresponding soluble CD63 agonist. Co-stimulation by the agent when plate-bound but not when soluble (or greater co-stimulation by the agent when plate-bound as compared to when soluble) indicates that the agent is a CD63 agonist, e.g., because the plate-bound agent is able to concentrate CD63 molecules on the surface of the cell, thereby facilitating signaling through CD63.

In certain aspects, the CD63 agonist is identified, e.g., using a suitable approach for screening small molecules (e.g., by screening a combinatorial library of small molecules), antibodies (e.g., by phage or yeast display of antibody libraries), ligands, or the like for the ability to bind CD63 with subsequent screening for the ability to agonize CD63.

Any of the CD63 agonists described herein may specifically bind to CD63. Such a CD63 agonist “specifically binds” to CD63 if it binds to or associates with CD63 with an affinity or Kₐ (that is, an equilibrium association constant of a particular binding interaction with units of 1/M) of, for example, greater than or equal to about 10⁵ M⁻¹. In certain embodiments, the CD63 agonist binds to CD63 with a Kₐ greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding refers to binding with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater. Alternatively, affinity may be defined as an equilibrium dissociation constant (K_{D}) of a particular binding interaction with units of M (e.g., 10⁻⁵ M to 10⁻¹³ M, or less). In certain aspects, specific binding means the CD63 agonist binds to CD63 with a K_{D} of less than or equal to about 10⁻⁵ M, less than or equal to about 10⁻⁶ M, less than or equal to about 10⁻⁷ M, less than or equal to about 10⁻⁸ M, or less than or equal to about 10⁻⁹ M, 10⁻¹⁰ M, 10⁻¹¹ M, or 10⁻¹² M or less. The binding affinity of the CD63 agonist for CD63 can be readily determined using conventional techniques, e.g., by competitive ELISA (enzyme-linked immunosorbent assay), by equilibrium dialysis, by using surface plasmon resonance (SPR) technology (e.g., the BIAcore 2000 instrument, using general procedures outlined by the manufacturer); by radioimmunoassay; or the like.

Methods are available for measuring the affinity of a candidate CD63 agonist for CD63 expressed on the surface of cells (e.g., T cells) using direct binding or competition binding assays. In a direct binding assay, the equilibrium binding constant (K_{D}) may be measured using a candidate CD63 agonist conjugated to a fluorophore or radioisotope, or a candidate CD63 agonist that contains an N- or C-terminal epitope tag for detection by a labeled antibody. If labels or tags are not feasible or desired, a competition binding assay can be used to determine the half-maximal inhibitory concentration (IC₅₀), the amount of unlabeled candidate CD63 agonist at which 50% of the maximal signal of the labeled competitor is detectable. A K_{D} value can then be calculated from the measured IC₅₀ value. Ligand depletion will be more pronounced when measuring high-affinity interactions over a lower concentration range, and can be avoided or minimized by decreasing the number of cells added in the experiment or by increasing the binding reaction volumes.

The CD63 agonist administered to the subject may vary. In certain aspects, the CD63 agonist is a small molecule. As used herein, a “small molecule” is a compound having a molecular weight of 1000 atomic mass units (amu) or less. In some embodiments, the small molecule is 750 amu or less, 500 amu or less, 400 amu or less, 300 amu or less, or 200 amu or less. In certain aspects, the small molecule is not made of repeating molecular units such as are present in a polymer. In some embodiments, the CD63 agonist is a small molecule known to bind CD63. In other aspects, a small molecule CD63 agonist is identified, e.g., using a suitable approach for screening small molecules, e.g., by screening a combinatorial library of small molecules.

In some embodiments, the CD63 agonist is a peptide or polypeptide. When the CD63 agonist is a peptide or polypeptide, the agonist may be a CD63 ligand. CD63 ligands of interest include, but are not limited to, tissue inhibitor of metalloproteinases-1 (TIMP-1; UniProtKB—P01033), integrin (31 (ITGB1; UniProtKB—Q5T3E5), or CD63-binding derivatives thereof, including CD63-binding fragments thereof. As used herein, a “derivative” peptide or polypeptide CD63 agonist (e.g., a ligand, an antibody, or the like) refers to a peptide or polypeptide that binds to and agonizes CD63 but has fewer or more amino acids than the wild-type/parental CD63 agonist, has one or more amino acid substitutions relative to the wild-type/parental CD63 agonist, or any combination thereof.

In certain aspects, the CD63 agonist is an antibody that specifically binds CD63. The terms “antibody” and “immunoglobulin” include antibodies or immunoglobulins of any isotype (e.g., IgG (e.g., IgG1, IgG2, IgG3 or IgG4), IgE, IgD, IgA, IgM, etc.), whole antibodies (e.g., antibodies composed of a tetramer which in turn is composed of two dimers of a heavy and light chain polypeptide); single chain antibodies; fragments of antibodies (e.g., fragments of whole or single chain antibodies) which retain specific binding to CD63, including, but not limited to, Fv, single chain Fv (scFv), Fab, F(ab′)2, Fab′, (scFv′)₂, and diabodies; chimeric antibodies; monoclonal antibodies, human antibodies, humanized antibodies (e.g., humanized whole antibodies, humanized antibody fragments, etc.); and fusion proteins including an antigen-binding portion of an antibody and a non-antibody protein or fragment thereof, e.g., an antibody Fc region or fragment thereof. The antibodies may be detectably labeled, e.g., with an in vivo imaging agent, or the like. The antibodies may be further conjugated to other moieties, such as, e.g., polyethylene glycol (PEG), etc. Fusion to an antibody Fc region (or a fragment thereof), conjugation to PEG, etc. may find use, e.g., for increasing serum half-life of the antibody upon administration to the subject.

The CD63 agonist may be a known CD63-binding agent. For example, in some embodiments, the CD63 agonist is an antibody known to specifically bind CD63 and determined to be an agonist of CD63. In one non-limiting example, the CD63 agonist may be an antibody having the binding properties of the MX-49.129.5 anti-human CD63 monoclonal antibody (Santa Cruz Biotechnology). For example, the CD63 agonist may be an antibody that competes for binding to human CD63 with the MX-49.129.5 anti-human CD63 antibody. Whether a first antibody “competes with” a second antibody for binding to the compound may be readily determined using competitive binding assays known in the art. Competing antibodies may be identified, for example, via an antibody competition assay. For example, a sample of a first antibody can be bound to a solid support. Then, a sample of a second antibody suspected of being able to compete with such first antibody is then added. One of the two antibodies is labelled. If the labeled antibody and the unlabeled antibody bind to separate and discrete sites on the compound, the labeled antibody will bind to the same level whether or not the suspected competing antibody is present. However, if the sites of interaction are identical or overlapping, the unlabeled antibody will compete, and the amount of labeled antibody bound to the antigen will be lowered. If the unlabeled antibody is present in excess, very little, if any, labeled antibody will bind.

For purposes of the present disclosure, competing antibodies are those that decrease the binding of an antibody to the compound by about 50% or more, about 60% or more, about 70% or more, about 80% or more, about 85% or more, about 90% or more, about 95% or more, or about 99% or more. Details of procedures for carrying out such competition assays are well known in the art and can be found, for example, in Harlow and Lane, Antibodies, A Laboratory Manual, Cold Spring Harbor Laboratory Press, Cold Spring Harbor, N.Y., 1988, 567-569, 1988, ISBN 0-87969-314-2. Such assays can be made quantitative by using purified antibodies. A standard curve may be established by titrating one antibody against itself, i.e., the same antibody is used for both the label and the competitor. The capacity of an unlabeled competing antibody to inhibit the binding of the labeled antibody to the plate may be titrated. The results may be plotted, and the concentrations necessary to achieve the desired degree of binding inhibition may be compared.

Antibodies that specifically bind CD63 (e.g., human CD63; UniProtKB—P08962) can be prepared using a wide variety of techniques known in the art including the use of hybridoma, recombinant, phage display technologies, or a combination thereof. For example, an antibody may be made and isolated using methods of phage display. Phage display is used for the high-throughput screening of protein interactions. Phages may be utilized to display antigen-binding domains expressed from a repertoire or combinatorial antibody library (e.g., human or murine). Phage expressing an antigen binding domain that binds CD63 can be selected or identified with CD63, e.g., using labeled CD63 bound or captured to a solid surface or bead. Phage used in these methods are typically filamentous phage including fd and M13 binding domains expressed from phage with Fab, Fv (individual Fv region from light or heavy chains) or disulfide stabilized Fv antibody domains recombinantly fused to either the phage gene III or gene VIII protein. Exemplary methods are set forth, for example, in U.S. Pat. No. 5,969,108, Hoogenboom, H. R. and Chames, Immunol. Today 2000, 21:371; Nagy et al. Nat. Med. 2002, 8:801; Huie et al., Proc. Natl. Acad. Sci. USA 2001, 98:2682; Lui et al., J. Mol. Biol. 2002, 315:1063, each of which is incorporated herein by reference. Several publications (e.g., Marks et al., Bio/Technology 1992, 10:779-783) have described the production of high affinity human antibodies by chain shuffling, as well as combinatorial infection and in vivo recombination as a strategy for constructing large phage libraries. In another embodiment, ribosomal display can be used to replace bacteriophage as the display platform (see, e.g., Hanes et al., Nat. Biotechnol. 2000, 18:1287; Wilson et al., Proc. Natl. Acad. Sci. USA 2001, 98:3750; or Irving et al., J. Immunol. Methods 2001, 248:31). Cell surface libraries may be screened for antibodies (Boder et al., Proc. Natl. Acad. Sci. USA 2000, 97:10701; Daugherty et al., J. Immunol. Methods 2000, 243:211). Such procedures provide alternatives to traditional hybridoma techniques for the isolation and subsequent cloning of monoclonal antibodies.

After phage selection, the antibody coding regions from the phage can be isolated and used to generate whole antibodies, including human antibodies, or any desired antigen binding fragment, and expressed in any desired host, including mammalian cells, insect cells, plant cells, yeast, and bacteria. For example, techniques to recombinantly produce Fv, scFv, Fab, F(ab′)₂, and Fab′ fragments may be employed using methods known in the art.

As summarized above, the subject to whom the therapeutically effective amount of a CD63 agonist is administered may be receiving a T cell activation therapy. In certain embodiments, the methods further include administering such a T cell activation therapy to the subject. According to some embodiments, the CD63 agonist is administered to the subject to potentiate the T cell activation therapy. By “T cell activation therapy” is meant a therapy that includes administration of a T cell activator to the subject. As used herein, a “T cell activator” is an agent that stimulates an immune response in a T cell or group of T cells. Such an immune response involves the engagement of the T cell receptor (TCR), present on the surface of a T cell, with a small peptide antigen non-covalently presented on the surface of an antigen presenting cell (APC) by a major histocompatibility complex (MHC; also referred to in humans as a human leukocyte antigen (HLA) complex). This engagement represents the immune system's targeting mechanism and is a requisite molecular interaction for T cell activation and effector function. Following epitope-specific cell targeting, the targeted T cells are activated through engagement of costimulatory proteins found on the APC with counterpart costimulatory proteins on the T cells. Both signals—epitope/TCR binding and engagement of APC costimulatory proteins with T cell costimulatory proteins—are required to drive T cell specificity and activation. The TCR is specific for a given epitope; however, the costimulatory protein is not epitope specific and instead is generally expressed on all T cells or on large T cell subsets.

Various assays can be utilized in order to determine whether an immune response has been stimulated in a T cell or group of T cells, i.e., whether a T cell or group of T cells has become “activated”. In certain aspects, stimulation of an immune response in T cells can be determined by measuring antigen-induced production of cytokines by T cells. In some embodiments, stimulation of an immune response in T cells can be determined by measuring antigen-induced production of IFNγ, IL-4, IL-2, IL-10, IL-17 and/or TNFα by T cells. In some embodiments, antigen-produced production of cytokines by T cells can be measured by intracellular cytokine staining followed by flow cytometry. In some embodiments, antigen-induced production of cytokines by T cells can be measured by surface capture staining followed by flow cytometry. In some embodiments, antigen-induced production of cytokines by T cells can be measured by determining cytokine concentration in supernatants of activated T cell cultures. In some embodiments, this can be measured by ELISA.

In some embodiments, antigen-produced production of cytokines by T cells can be measured by ELISPOT assay. In general, ELISPOT assays employ a technique very similar to the sandwich enzyme-linked immunosorbent assay (ELISA) technique. An antibody (e.g. monoclonal antibody, polyclonal antibody, etc.) is coated aseptically onto a PVDF (polyvinylidene fluoride)-backed microplate. Antibodies are chosen for their specificity for the cytokine in question. The plate is blocked (e.g. with a serum protein that is non-reactive with any of the antibodies in the assay). Cells of interest are plated out at varying densities, along with antigen or mitogen, and then placed in a humidified 37° C. CO2 incubator for a specified period of time. Cytokine secreted by activated cells is captured locally by the coated antibody on the high surface area PVDF membrane. After washing the wells to remove cells, debris, and media components, a secondary antibody (e.g., a biotinylated polyclonal antibody) specific for the cytokine is added to the wells. This antibody is reactive with a distinct epitope of the target cytokine and thus is employed to detect the captured cytokine. Following a wash to remove any unbound biotinylated antibody, the detected cytokine is then visualized using an avidin-HRP, and a precipitating substrate (e.g., AEC, BCIP/NBT). The colored end product (a spot, usually a blackish blue) typically represents an individual cytokine-producing cell. Spots can be counted manually (e.g., with a dissecting microscope) or using an automated reader to capture the microwell images and to analyze spot number and size. In some embodiments, each spot correlates to a single cytokine-producing cell.

In some instances, T cells activated by the T cell activator are specific for an epitope present on abnormally proliferative cells underlying the cellular proliferative disorder (e.g., cancer cells in an individual having cancer), and contacting such T cells with the T cell activator increases cytotoxic activity of the T cells toward the abnormally proliferating cells, increases the number of such epitope-specific T cells, or a combination thereof. In some embodiments, the increase in cytotoxic activity is further potentiated by the CD63 agonist.

A wide variety of agents may be employed as the T cell activator. In certain embodiments, the T cell activator is an immune checkpoint inhibitor. As used herein, an “immune checkpoint inhibitor” is any agent (e.g., small molecule, nucleic acid, protein (e.g., antibody)) that prevents the suppression of any component in the immune system such as MHC class presentation, T cell presentation and/or differentiation, any cytokine, chemokine or signaling for immune cell proliferation and/or differentiation. According to some embodiments, the immune checkpoint inhibitor is selected from a cytotoxic T-lymphocyte-associated antigen 4 (CTLA-4) inhibitor, a programmed cell death-1 (PD-1) inhibitor, a programmed cell death ligand-1 (PD-L1) inhibitor, a lymphocyte activation gene-3 (LAG-3) inhibitor, a T-cell immunoglobulin domain and mucin domain 3 (TIM-3) inhibitor, an indoleamine (2,3)-dioxygenase (IDO) inhibitor, a T cell immunoreceptor with Ig and ITIM domains (TIGIT) inhibitor, a V-domain Ig suppressor of T cell activation (VISTA) inhibitor, a B7-H3 inhibitor, and any combination thereof.

According to some embodiments, the T cell activator is an agonist of a T cell co-stimulatory receptor. Non-limiting examples of such agonists include an OX40 agonist, a glucocorticoid-induced TNFR-related protein (GITR) agonist, a CD137 agonist, and a CD40 agonist.

In certain embodiments, the T cell activator is an antagonist of a T cell inhibitory signal.

Inhibitory antibodies to T cell inhibitors such as PD-1 or CTLA-4 increase the activation of T cells. These inhibitory T cell signals play a role in the natural tolerance to auto-antigens but also can play a role to limit T cell function against tumor antigens. Hence, when blocking Abs are administered to tumor-bearing hosts they increase T cell activation/responses against tumors leading to increased immune-mediated tumor destruction.

According to some embodiments, the T cell activator is a cytokine. Cytokines of interest in the context of the present disclosure are those that promote T cell activation (e.g., IL-1, and the like), promote proliferation of activated T cells (e.g., IL-2, and the like), etc. Non-limiting examples of cytokines that may be administered with the CD63 agonist include IL-1, IL-2, IL-4, IL-15, and any combination thereof.

In certain embodiments, the T cell activator is an agent that blocks immune suppressive cytokines. A non-limiting example of such a T cell activator is a TGF-β receptor inhibitor.

According to some embodiments, the T cell activator is an antagonist of an inhibitory immune receptor. In certain embodiments, such an antagonist binds directly to the inhibitory immune receptor, thereby blocking activation of the inhibitory immune receptor, e.g., by preventing binding of the receptor to its ligand. In other aspects, such an antagonist binds to the ligand of an inhibitory immune receptor, thereby blocking activation of the inhibitory immune receptor by preventing binding of the receptor to its ligand. Antagonists of inhibitory immune receptors that may be administered with the CD63 agonist include, but are not limited to, TGF-β.

In certain embodiments, the subject to whom the CD63 agonist is administered is receiving an innate immune system stimulator therapy. According to some embodiments, the methods further include administering the innate immune system stimulator therapy to the subject. Optionally, at the time of the administering (e.g., the initial administration of the CD63 agonist, the innate immune system stimulator, or both (if present in a single formulation)), abnormally proliferating cells of the cell proliferative disorder are not suspected of exhibiting overexpression of CD63.

As used herein, an “innate immune system stimulator” is an agent that stimulates an innate immune system response in the subject. Examples of agents that may be employed to stimulate the innate immune system include, but are not limited to, agents that bind to one or more Toll-like receptors (TLRs), e.g., one or more of TLR1-TLR9. Most mammalian species have 10-13 types of TLRs and each receptor recognizes specific ligands and induces a wide array of inflammatory cascades.

In some embodiments, the innate immune system stimulator is an agent that includes unmethylated CpG dinucleotides. It is now understood that the immune stimulatory effects of bacterial DNA are a result of the presence of unmethylated CpG dinucleotides in particular base contexts (CpG motifs), which are common in bacterial DNA, but methylated and underrepresented in vertebrate DNA (Krieg et al, 1995 Nature 374:546-549; Krieg, 1999 Biochim. Biophys. Acta 93321:1-10). The immune stimulatory effects of bacterial DNA can be mimicked with synthetic oligodeoxynucleotides (ODN) containing these CpG motifs. Such CpG ODN have highly stimulatory effects on human and murine leukocytes, inducing B cell proliferation; cytokine and immunoglobulin secretion; natural killer (NK) cell lytic activity and IFN-γ secretion; and activation of dendritic cells (DCs) and other antigen presenting cells to express costimulatory molecules and secrete cytokines, especially the Thi-like cytokines that are important in promoting the development of Thi-like T cell responses. These immune stimulatory effects of native phosphodiester backbone CpG ODN are highly CpG specific in that the effects are dramatically reduced if the CpG motif is methylated, changed to a GpC, or otherwise eliminated or altered (Krieg et al, 1995 Nature 374:546-549; Hartmann et al, 1999 Proc. Natl. Acad. Sci USA 96:9305-10).

The CD63 agonist and, if also administered, a T cell activator and/or innate immune system stimulator, are administered in a therapeutically effective amount. By “therapeutically effective amount” is meant a dosage sufficient to produce a desired result, e.g., an amount sufficient to effect beneficial or desired therapeutic (including preventative) results, such as a reduction in a symptom of the proliferative disorder, as compared to a control. When the cell proliferative disorder is cancer, in some embodiments, the therapeutically effective amount is sufficient to slow the growth of a tumor, reduce the size of a tumor, and/or the like. An effective amount can be administered in one or more administrations.

When the methods include administering a combination of the CD63 agonist and a second agent (e.g., a T cell activator and/or innate immune system stimulator), the CD63 agonist and the second agent may be administered concurrently (e.g., in the same or separate formulations), sequentially, or both. For example, according to certain embodiments, the second agent is administered to the individual prior to administration of the CD63 agonist, concurrently with administration of the CD63 agonist, or both. In some embodiments, the CD63 agonist is administered to the individual prior to administration of the second agent, concurrently with administration of the second agent, or both.

According to some embodiments, the methods include administering the CD63 agonist to a subject to whom a T cell activator (e.g., immune checkpoint blocker (such as a PD1 inhibitor, etc.) or other T cell activator) has already been administered, and the CD63 agonist is administered to potentiate the efficacy of the prior administered T cell activator. A non-limiting example of such potentiation is demonstrated in the Experimental section below.

In certain embodiments, the one or more agents are administered according to a dosing regimen approved for individual use. In some embodiments, the administration of the CD63 agonist permits the second agent to be administered according to a dosing regimen that involves one or more lower and/or less frequent doses, and/or a reduced number of cycles as compared with that utilized when the second agent is administered without administration of the CD63 agonist. In some embodiments, the administration of the second agent permits the CD63 agonist to be administered according to a dosing regimen that involves one or more lower and/or less frequent doses, and/or a reduced number of cycles as compared with that utilized when the CD63 agonist is administered without administration of the second agent.

As noted above, in certain embodiments, one or more doses of the CD63 agonist and second agent are administered at the same time; in some such embodiments, such agents may be administered present in the same pharmaceutical composition. In some embodiments, however, the CD63 agonist and second agent are administered to the individual in different compositions and/or at different times. For example, the CD63 agonist may be administered prior to administration of the second agent (e.g., in a particular cycle). Alternatively, the second agent may be administered prior to administration of the CD63 agonist (e.g., in a particular cycle). The second agent to be administered may be administered a period of time that starts at least 1 hour, 3 hours, 6 hours, 12 hours, 24 hours, 48 hours, 72 hours, or up to 5 days or more prior to or after the administration of the CD63 agonist.

In one example, the CD63 agonist is administered to the individual for a desirable period of time prior to administration of the second agent. In certain aspects, such a regimen “primes” the immune system, e.g., for T cell activation by the T cell activator and/or innate immune system stimulation by the innate immune system stimulator. In another example, the second agent is administered to the individual for a desirable period of time prior to administration of the CD63 agonist. In certain aspects, such a regimen “primes” the immune system for T cell activation by the CD63 agonist.

In some embodiments, administration of one agent is specifically timed relative to administration of another agent. For example, in some embodiments, a first agent is administered so that a particular effect is observed (or expected to be observed, for example based on population studies showing a correlation between a given dosing regimen and the particular effect of interest).

In certain aspects, desired relative dosing regimens for agents administered in combination may be assessed or determined empirically, for example using ex vivo, in vivo and/or in vitro models; in some embodiments, such assessment or empirical determination is made in vivo, in a patient population (e.g., so that a correlation is established), or alternatively in a particular subject of interest.

By way of example, the CD63 agonist may be administered a period of time after administration of a T cell activator and/or innate immune system stimulator. The period of time may be selected to be correlated with an increase in T cell activation and/or innate immune system stimulation by the T cell activator and/or innate immune system stimulator, respectively. In some embodiments, the relevant period of time permits (e.g., is correlated with) T cell activation and/or innate immune system stimulation that is 10% or more, 20% or more, 30% or more, 40% or more, 50% or more, 60% or more, 70% or more, 80% or more, 90% or more, 100% or more, 150% or more, 200% or more, 250% or more, 300% or more, 350% or more, 400% or more, 450% or more, or 500% or more, than that observed prior to (or at the moment of) the administration of a T cell activator and/or innate immune system stimulator, respectively.

In some embodiments, the CD63 agonist and second agent are administered according to an intermittent dosing regimen including at least two cycles. Where two or more agents are administered in combination, and each by such an intermittent, cycling, regimen, individual doses of different agents may be interdigitated with one another. In certain aspects, one or more doses of the second agent is administered a period of time after a dose of the first agent. In some embodiments, each dose of the second agent is administered a period of time after a dose of the first agent. In certain aspects, each dose of the first agent is followed after a period of time by a dose of the second agent. In some embodiments, two or more doses of the first agent are administered between at least one pair of doses of the second agent; in certain aspects, two or more doses of the second agent are administered between al least one pair of doses of the first agent. In some embodiments, different doses of the same agent are separated by a common interval of time; in some embodiments, the interval of time between different doses of the same agent varies. In certain aspects, different doses of the different agents are separated from one another by a common interval of time; in some embodiments, different doses of the different agents are separated from one another by different intervals of time.

One exemplary protocol for interdigitating two intermittent, cycled dosing regimens (e.g., for potentiating the effect of the T cell activator, CD63 agonist, or both), may include: (a) a first dosing period during which a therapeutically effective amount a first agent is administered to an individual; (b) a first resting period; (c) a second dosing period during which a therapeutically effective amount of a second agent and, optionally, a third agent, is administered to the individual; and (d) a second resting period.

In some embodiments, the first resting period and second resting period may correspond to an identical number of hours or days. Alternatively, in some embodiments, the first resting period and second resting period are different, with either the first resting period being longer than the second one or, vice versa. In some embodiments, each of the resting periods corresponds to 120 hours, 96 hours, 72 hours, 48 hours, 24 hours, 12 hours, 6 hours, 30 hours, 1 hour, or less. In some embodiments, if the second resting period is longer than the first resting period, it can be defined as a number of days or weeks rather than hours (for instance 1 day, 3 days, 5 days, 1 week, 2, weeks, 4 weeks or more).

If the first resting period's length is determined by existence or development of a particular biological or therapeutic event (e.g., increased T cell activation), then the second resting period's length may be determined on the basis of different factors, separately or in combination. Exemplary such factors may include type and/or stage of a cancer against which the agents are administered; identity and/or properties (e.g., pharmacokinetic properties) of the first agent, and/or one or more features of the patient's response to therapy with the first agent. In some embodiments, length of one or both resting periods may be adjusted in light of pharmacokinetic properties (e.g., as assessed via plasma concentration levels) of one or the other (or both) of the administered agents. For example, a relevant resting period might be deemed to be completed when plasma concentration of the relevant agent is below about 1 μg/ml, 0.1 μg/ml, 0.01 μg/ml or 0.001 μg/ml, optionally upon evaluation or other consideration of one or more features of the individual's response.

In certain aspects, the number of cycles for which a particular agent is administered may be determined empirically. Also, in some embodiments, the precise regimen followed (e.g., number of doses, spacing of doses (e.g., relative to each other or to another event such as administration of another therapy), amount of doses, etc.) may be different for one or more cycles as compared with one or more other cycles.

The CD63 agonist, and if also administered, a second agent (e.g., a T cell activator and/or innate immune system stimulator), may be administered via a route of administration independently selected from oral, parenteral (e.g., by intravenous, intra-arterial, subcutaneous, intramuscular, or epidural injection), topical, or nasal administration. According to certain embodiments, the CD63 agonist and a second agent are both administered parenterally, either concurrently (in the same pharmaceutical composition or separate pharmaceutical compositions) or sequentially.

As described above, aspects of the present disclosure include methods for treating a cell proliferative disorder (e.g., cancer). By treatment is meant at least an amelioration of one or more symptoms associated with the cell proliferative disorder of the individual, where amelioration is used in a broad sense to refer to at least a reduction in the magnitude of a parameter, e.g. symptom, associated with the cell proliferative disorder (e.g., cancer) being treated. As such, treatment also includes situations where the cell proliferative disorder, or at least one or more symptoms associated therewith, are completely inhibited, e.g., prevented from happening, or stopped, e.g., terminated, such that the individual no longer suffers from the cell proliferative disorder, or at least the symptoms that characterize the cell proliferative disorder. When the subject has cancer, “treating” the cancer may include slowing the rate of tumor growth in the subject.

Compositions

As summarized above, aspects of the present disclosure include pharmaceutical compositions. In certain embodiments, a pharmaceutical composition of the present disclosure includes a CD63 agonist (e.g., any of the CD63 agonists described above), and a pharmaceutically acceptable excipient. According to some embodiments, a pharmaceutical composition of the present disclosure includes a CD63 agonist (e.g., any of the CD63 agonists described above), a T cell activator (e.g., any of the T cell activators described above), and a pharmaceutically acceptable excipient. In certain embodiments, a pharmaceutical composition of the present disclosure includes a CD63 agonist (e.g., any of the CD63 agonists described above), an innate immune system stimulator (e.g., any of the innate immune system stimulators described above), and a pharmaceutically acceptable excipient.

As will be appreciated, the pharmaceutical compositions of the present disclosure may include any of the agents and features described above in the section relating to the subject methods, which are incorporated but not reiterated in detail herein for purposes of brevity.

In some embodiments, the CD63 agonist present in the pharmaceutical composition is a small molecule. In certain aspects, the CD63 agonist present in the pharmaceutical composition is a peptide or polypeptide. When the CD63 agonist is a peptide or polypeptide, the CD63 agonist may be a CD63 ligand, an antibody that specifically binds CD63, or the like. When the CD63 agonist is an antibody, the antibody may be selected from an IgG, Fv, scFv, Fab, F(ab′)2, and Fab′.

When a pharmaceutical composition of the present disclosure includes a T cell activator, in certain embodiments, the T cell activator is an immune checkpoint inhibitor. When the T cell activator is an immune checkpoint inhibitor, according to some embodiments, the immune checkpoint inhibitor is selected from a cytotoxic T-lymphocyte-associated antigen 4 (CTLA-4) inhibitor, a programmed cell death-1 (PD-1) inhibitor, a programmed cell death ligand-1 (PD-L1) inhibitor, a lymphocyte activation gene-3 (LAG-3) inhibitor, a T-cell immunoglobulin domain and mucin domain 3 (TIM-3) inhibitor, an indoleamine (2,3)-dioxygenase (IDO) inhibitor, a T cell immunoreceptor with Ig and ITIM domains (TIGIT) inhibitor, a V-domain Ig suppressor of T cell activation (VISTA) inhibitor, a B7-H3 inhibitor, and any combination thereof.

When a pharmaceutical composition of the present disclosure includes a T cell activator, in certain embodiments, the T cell activator is an agonist of a T cell co-stimulatory receptor, such as but not limited to, an OX40 agonist, a glucocorticoid-induced TNFR-related protein (GITR) agonist, a CD137 agonist, a CD40 agonist, and/or the like.

When a pharmaceutical composition of the present disclosure includes a T cell activator, in some embodiments, the T cell activator is an antagonist of a T cell inhibitory signal, a cytokine, an agent that blocks immune suppressive cytokines (e.g., a TGF-β receptor inhibitor), and/or an antagonist of an inhibitory immune receptor. In some embodiments, a pharmaceutical composition of the present disclosure includes the CD63 agonist and a combination (that is, two or more) of T cell activators, including but not limited to any combination of the T cell activators described herein.

The CD63 agonist, T cell activator, or both, can be incorporated into a variety of formulations for therapeutic administration. More particularly, the agent(s) (that is, CD63 agonist and/or T cell activator) can be formulated into pharmaceutical compositions by combination with appropriate, pharmaceutically acceptable excipients or diluents, and may be formulated into preparations in solid, semi-solid, liquid or gaseous forms, such as tablets, capsules, powders, granules, ointments, solutions, injections, inhalants and aerosols.

Formulations of the agents for administration to the subject (e.g., suitable for human administration) are generally sterile and may further be free of detectable pyrogens or other contaminants contraindicated for administration to a patient according to a selected route of administration.

In pharmaceutical dosage forms, the agent(s) can be administered in the form of their pharmaceutically acceptable salts, or they may also be used alone or in appropriate association, as well as in combination, with other pharmaceutically active compounds. The following methods and carriers/excipients are merely examples and are in no way limiting.

For oral preparations, the agent(s) can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agent(s) can be formulated for parenteral (e.g., intravenous, intra-arterial, intraosseous, intramuscular, intracerebral, intracerebroventricular, intrathecal, subcutaneous, etc.) administration. In certain aspects, the agent(s) are formulated for injection by dissolving, suspending or emulsifying the agent(s) in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Pharmaceutical compositions that include the agent(s) may be prepared by mixing the agent(s) having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition may be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents may be used for the production of pharmaceutical compositions for parenteral administration.

An aqueous formulation of the agent(s) may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included to modulate the tonicity of the formulation. Example tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 mM.

A surfactant may also be added to the formulation to reduce aggregation and/or minimize the formation of particulates in the formulation and/or reduce adsorption. Example surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80®). Examples of suitable polyethylene-polypropylene copolymers are those sold under the names Pluronic® F68 or Poloxamer 188®. Examples of suitable Polyoxyethylene alkyl ethers are those sold under the trademark Brij™. Example concentrations of surfactant may range from about 0.001% to about 1% w/v.

A lyoprotectant may also be added in order to protect the CD63 agonist and/or T cell activator against destabilizing conditions during a lyophilization process. For example, known lyoprotectants include sugars (including glucose and sucrose); polyols (including mannitol, sorbitol and glycerol); and amino acids (including alanine, glycine and glutamic acid). Lyoprotectants can be included in an amount of about 10 mM to 500 nM.

In some embodiments, the pharmaceutical composition includes the CD63 agonist and/or T cell activator, and one or more of the above-identified components (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

Kits

As summarized above, the present disclosure provides kits. The kits find use, e.g., in practicing the methods of the present disclosure. According to some embodiments, a subject kit includes a pharmaceutical composition that includes a CD63 agonist, e.g., any of the CD63 agonists described elsewhere herein. In certain embodiments, provided are kits that include any of the pharmaceutical compositions described herein, including any of the pharmaceutical compositions described above in the section relating to the compositions of the present disclosure. Kits of the present disclosure may include instructions for administering the pharmaceutical composition to a subject having a cell proliferative disorder, e.g., cancer. According to some embodiments, a kit of the present disclosure includes instructions for administering the pharmaceutical composition to the subject to enhance a T cell response to abnormally proliferating cells of the cell proliferative disorder. The instructions may include instructions for administering the pharmaceutical composition to the subject independent of the level of expression of CD63 on the abnormally proliferating cells of the cell proliferative disorder. In certain embodiments, a kit of the present disclosure includes instructions for administering the pharmaceutical composition to a subject having a cell proliferative disorder in which abnormally proliferating cells of the cell proliferative disorder are not suspected of exhibiting expression or overexpression of CD63. According to some embodiments, a kit of the present disclosure includes instructions for administering the pharmaceutical composition to a subject receiving a T cell activation therapy, e.g., to potentiate the T cell activation therapy.

In some embodiments, kits are provided that include a CD63 agonist, a T cell activator, and instructions for administering the CD63 agonist and T cell activator to a subject having a cell proliferative disorder, e.g., cancer. The instructions may further include any of the instructions for the kits described above, e.g., for administering the pharmaceutical composition to a subject having a cell proliferative disorder to enhance a T cell response to abnormally proliferating cells of the cell proliferative disorder (e.g., cancer), for administering the pharmaceutical composition to the subject independent of the level of expression of CD63 on the abnormally proliferating cells of the cell proliferative disorder, and/or the like. The CD63 agonist and T cell activator may be present in the same container, or may be present in separate containers. When the CD63 agonist and T cell activator are present in separate containers, the kit may include instructions to administer the CD63 agonist prior to and/or subsequent to administering the T cell activator (e.g., immune checkpoint blocker (such as a PD1 inhibitor, etc.) or other T cell activator) to potentiate the efficacy of the T cell activator.

The subject kits may include a quantity of the compositions, present in unit dosages, e.g., ampoules, or a multi-dosage format. As such, in certain embodiments, the kits may include one or more (e.g., two or more) unit dosages (e.g., ampoules) of a composition that includes a CD63 agonist, a T cell activator, or both. The term “unit dosage”, as used herein, refers to physically discrete units suitable as unitary dosages for human and animal subjects, each unit containing a predetermined quantity of the composition calculated in an amount sufficient to produce the desired effect. The amount of the unit dosage depends on various factors, such as the particular CD63 agonist and/or T cell activator employed, the effect to be achieved, and the pharmacodynamics associated with the CD63 agonist and/or T cell activator, in the subject. In yet other embodiments, the kits may include a single multi dosage amount of the composition.

As will be appreciated, the kits of the present disclosure may include any of the agents and features described above in the section relating to the subject methods and compositions, which are incorporated but not reiterated in detail herein for purposes of brevity.

In some embodiments, the CD63 agonist present in the kit is a small molecule. In certain aspects, the CD63 agonist present in the kit is a peptide or polypeptide. When the CD63 agonist is a peptide or polypeptide, the CD63 agonist may be a CD63 ligand, an antibody that specifically binds CD63, or the like. When the CD63 agonist is an antibody, the antibody may be selected from an IgG, Fv, scFv, Fab, F(ab′)2, and Fab′.

When a kit of the present disclosure includes a T cell activator, in certain embodiments, the T cell activator is an immune checkpoint inhibitor. When the T cell activator is an immune checkpoint inhibitor, according to some embodiments, the immune checkpoint inhibitor is selected from a cytotoxic T-lymphocyte-associated antigen 4 (CTLA-4) inhibitor, a programmed cell death-1 (PD-1) inhibitor, a programmed cell death ligand-1 (PD-L1) inhibitor, a lymphocyte activation gene-3 (LAG-3) inhibitor, a T-cell immunoglobulin domain and mucin domain 3 (TIM-3) inhibitor, an indoleamine (2,3)-dioxygenase (IDO) inhibitor, a T cell immunoreceptor with Ig and ITIM domains (TIGIT) inhibitor, a V-domain Ig suppressor of T cell activation (VISTA) inhibitor, a B7-H3 inhibitor, and any combination thereof.

When a kit of the present disclosure includes a T cell activator, in certain embodiments, the T cell activator is an agonist of a T cell co-stimulatory receptor, such as but not limited to, an OX40 agonist, a glucocorticoid-induced TNFR-related protein (GITR) agonist, a CD137 agonist, a CD40 agonist, and/or the like.

When a kit of the present disclosure includes a T cell activator, in some embodiments, the T cell activator is an antagonist of a T cell inhibitory signal, a cytokine, an agent that blocks immune suppressive cytokines (e.g., a TGF-β receptor inhibitor), and/or an antagonist of an inhibitory immune receptor. In some embodiments, a kit of the present disclosure includes the CD63 agonist and a combination (that is, two or more) of T cell activators, including but not limited to a combination of any of the T cell activators described herein.

In some embodiments, the CD63 agonist present in the kit is a small molecule. In certain aspects, the CD63 agonist present in the kit is a peptide or polypeptide. When the CD63 agonist is a peptide or polypeptide, the CD63 agonist may be a CD63 ligand, an antibody that specifically binds CD63, or the like. When the CD63 agonist is an antibody, the antibody may be selected from an IgG, Fv, scFv, Fab, F(ab′)2, and Fab′.

When a kit of the present disclosure includes a T cell activator, in certain aspects, the T cell activator is an immune checkpoint inhibitor. Immune checkpoint inhibitors of interest include, but are not limited to, an agonist of a T cell co-stimulatory receptor, an antagonist of a T cell inhibitory signal, and/or the like. In some embodiments, the immune checkpoint inhibitor is selected from a cytotoxic T-lymphocyte-associated antigen 4 (CTLA-4) inhibitor, a programmed cell death-1 (PD-1) inhibitor, a programmed cell death ligand-1 (PD-L1) inhibitor, a lymphocyte activation gene-3 (LAG-3) inhibitor, a T-cell immunoglobulin domain and mucin domain 3 (TIM-3) inhibitor, an indoleamine (2,3)-dioxygenase (IDO) inhibitor, an OX40 agonist, a glucocorticoid-induced TNFR-related protein (GITR) agonist, a CD137 agonist, and a CD40 agonist. When a kit of the present disclosure includes a T cell activator, in certain aspects, the T cell activator is a cytokine. In some embodiments, when a kit of the present disclosure includes a T cell activator, the T cell activator is an antagonist of an inhibitory immune receptor. In certain aspects, a kit of the present disclosure includes a combination (that is, two or more) of any of the T cell activators described herein.

Components of the kits may be present in separate containers, or multiple components may be present in a single container. A suitable container includes a single tube (e.g., vial), ampoule, one or more wells of a plate (e.g., a 96-well plate, a 384-well plate, etc.), or the like.

The instructions (e.g., instructions for use (IFU)) included in the kits may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

The following examples are offered by way of illustration and not by way of limitation.

### EXPERIMENTAL

### Example 1—Co-Stimulation of Human T Cells Via CD63 and CD3

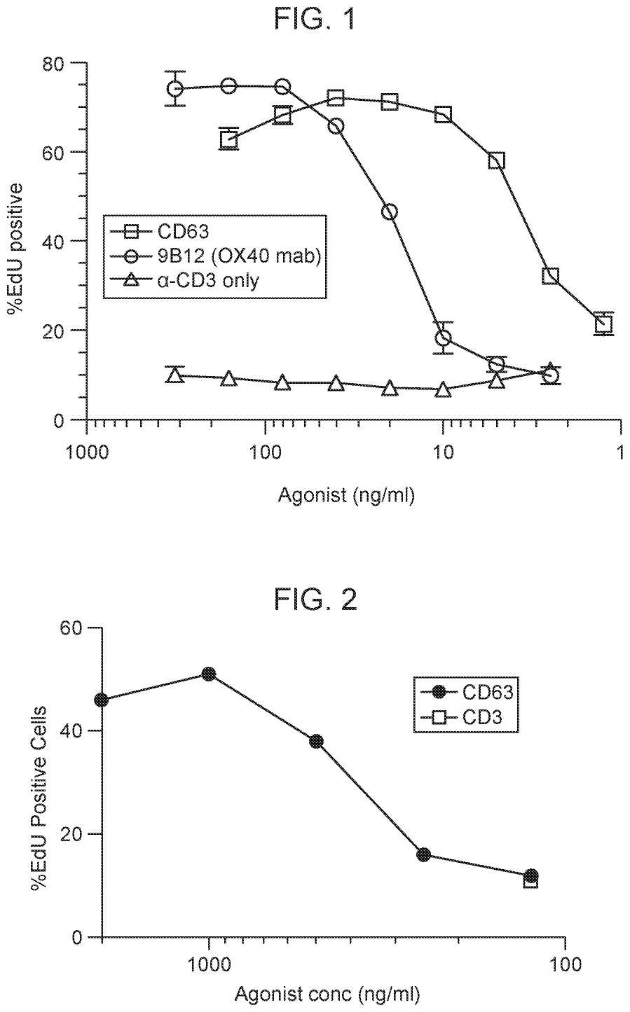

The binding agents to TCR (such as anti-CD3 antibodies) and to a potential co-stimulation target may be provided in tandem to purified T cells. The resulting T cell proliferation may be quantified and used to gauge the efficacy of the co-stimulation target. Shown in FIG. 1 is a graph depicting the results of T cell co-stimulation assays using anti-CD3 along with an anti-human CD63 co-stimulatory antibody (clone MX-49.129.5). Anti-OX40 (Providence, 9B12) is used as a positive control and 1% BSA/DPBS as a negative control.

CD4+ T cells were purified from frozen PBMCs and resuspended in RPMI media supplemented with penicillin, streptomycin, glutamine, non-essential amino acids, HEPES buffer, and sodium pyruvate. PHA-L (2 μg/ml) and IL2 (60 IU/ml) were added to the media to activate the cells, which were then incubated at 37° under 5% CO2 for 96 hours. Ninety-six-well plates were coated with goat anti-mouse IgG and then blocked with BSA. Mouse anti-human CD3 (OKT3) was prepared at 2 ng/ml in 1% BSA/DPBS, and added to the plates. Mouse anti-human CD63 was then added in serial, two-fold dilutions between 160 ng/ml and 1.25 ng/ml or mouse anti-human OX40 between 320 and 2.5 ng/ml. Activated CD4+ T cells were then added to each well at 50,000 cells/well in 200 μl of media, and then incubated at 37° for 48 hours. Cells were then labeled with 5-ethynyl-2′-deoxyuridine (EdU) at a final concentration of 10 μM, using the Click-it DNA labeling kit (Invitrogen) as per the manufacturer's instructions for 18 hours. Cells were then treated with live-dead stain (E450; eBiosciences) and developed using the Alex647 agent (Invitrogen). Proliferating cells were then quantified via flow cytometry.

As shown in FIG. 1, the MX-49.129.5 anti-CD63 clone stimulated proliferation of CD4+ T cells in a dose-dependent manner, with proliferation observed at concentrations as low as 2.5 ng/ml of anti-CD63. In comparison, proliferation via the classic CD4 T cell co-stimulator, OX40, was observed at concentrations as low as 10-20 ng/ml.

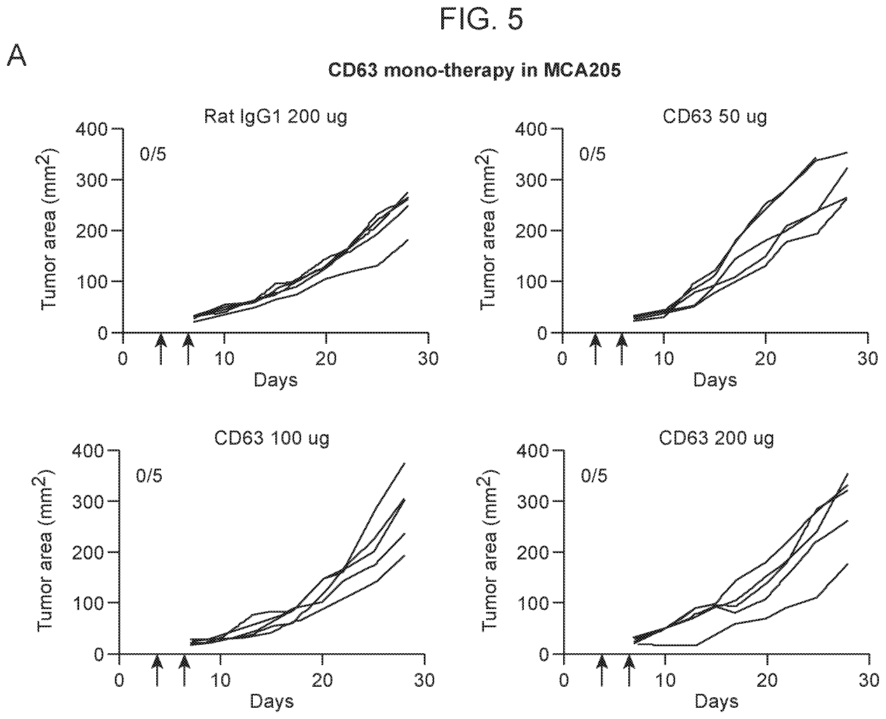

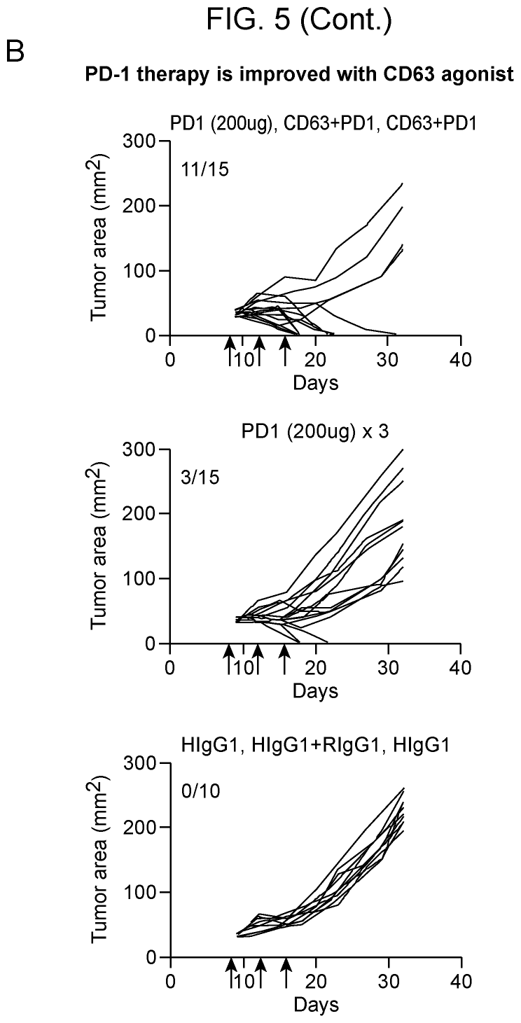

In a second study using the anti-mouse CD63 antibody MA5-24169 (ThermoFisher Scientific) (the antibody employed in the in vivo mouse studies presented below and shown in FIG. 5), mouse splenocytes from a C57131/6 mouse were enriched for CD4 T cells using a negative selection kit. Cells were treated with CD3 250 ng/ml+CD63 agonist (the MA5-24169 antibody) at a range from 0.125-2 μg/ml. Cells were then labeled with 5-ethynyl-2′-deoxyuridine (EdU) at a final concentration of 10 μM, using the Click-it DNA labeling kit (Invitrogen) as per the manufacturer's instructions. Cells were treated with IL-2 (50 U) and harvested at day 3. Cells were then treated with live-dead stain (E450; eBiosciences) and developed using the Alex647 agent (Invitrogen). Proliferating cells were then quantified via flow cytometry. Results are shown in FIG. 2.

The data indicates that both the MX-49.129.5 and MA5-24169 anti-CD63 antibodies result in dose-dependent co-stimulation/stimulation (mitogenesis) of the CD4+ T cells when co-cultured with anti-CD3, thereby demonstrating that co-stimulation with a CD63 agonist increases T cell proliferation.

### Example 2—CD63 Expression Levels in Cancer

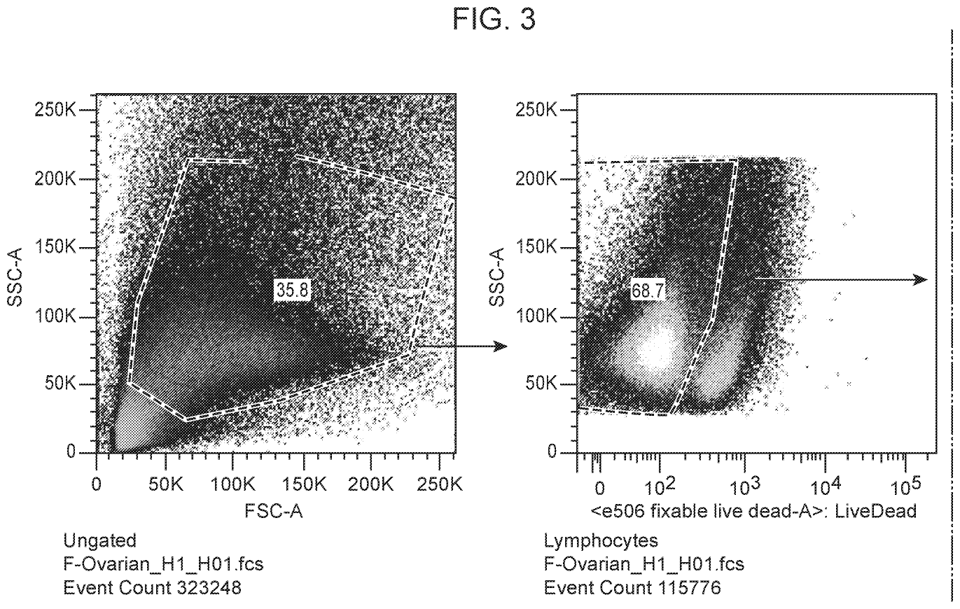

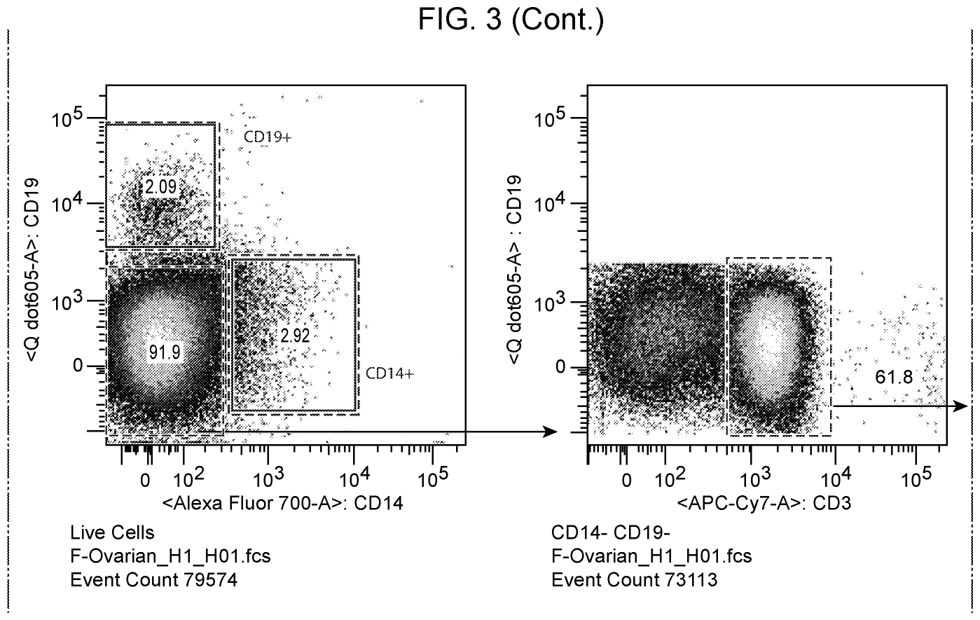

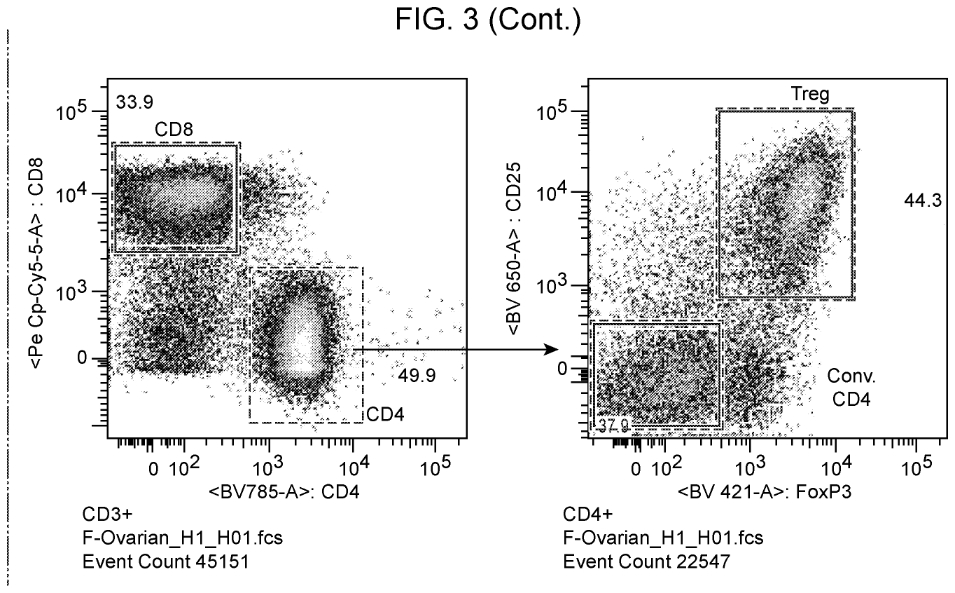

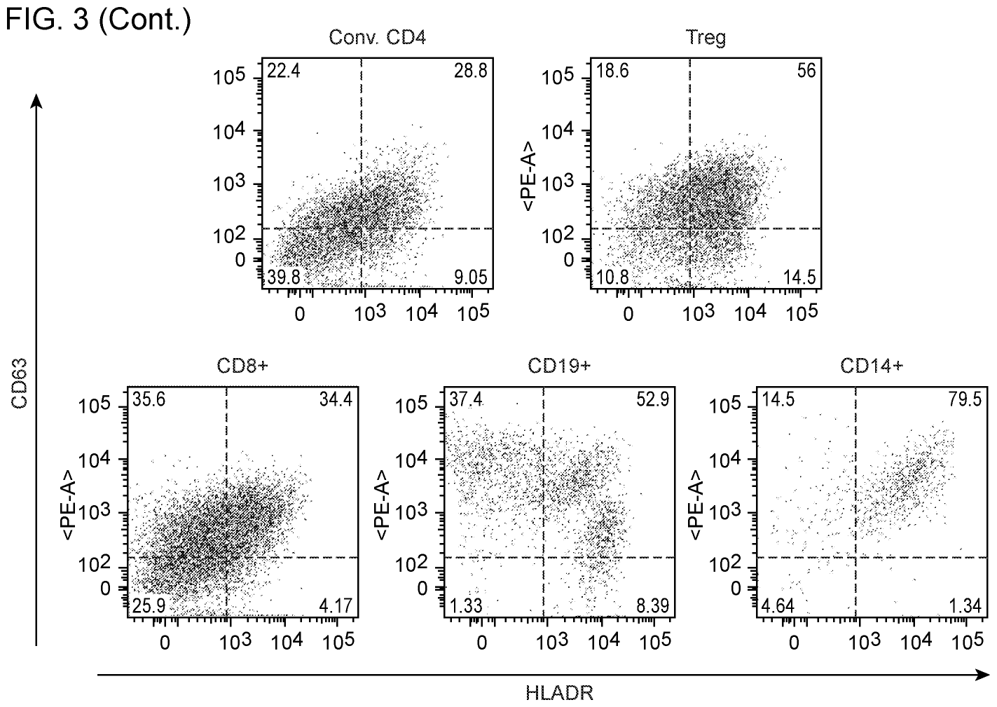

The expression levels of CD63 on T cells in the tumor context were assessed by flow cytometry. Shown in FIG. 3 is flow cytometry data showing CD63 expression levels on T cells in human ovarian cancer. Increases in CD63 levels on T cells correlated with increased HLA-DR expression on conventional CD4+, Tregs, CD8+ and CD14+ cells in the tumor.

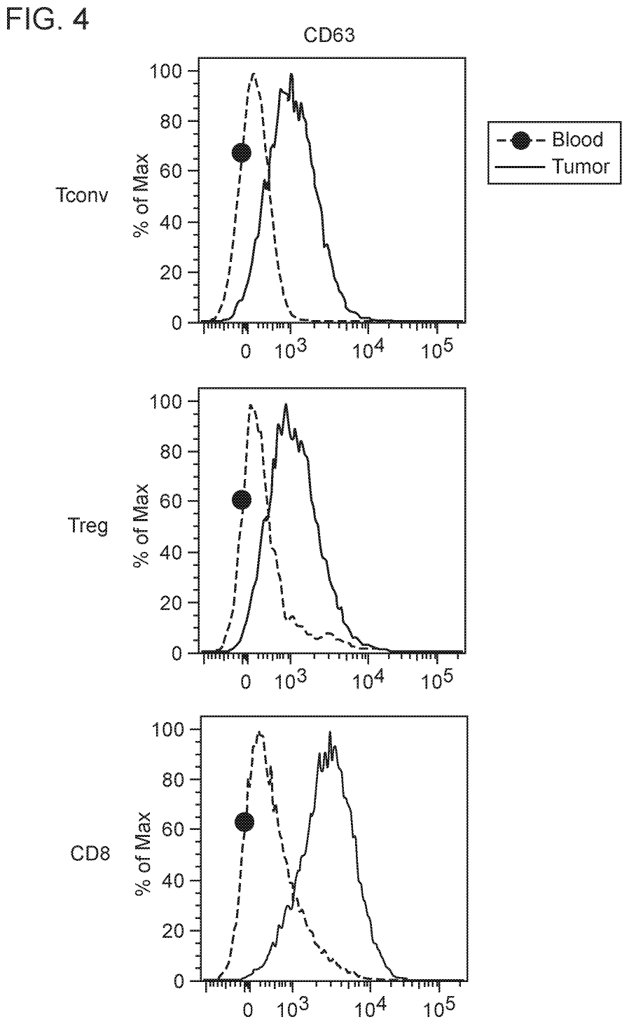

Shown in FIG. 4 is flow cytometry data showing CD63 expression levels on tumor infiltrating lymphocytes (TILs) relative to blood in a human head and neck cancer patient. An upregulation of CD63 is seen in the TILs as compared to blood from the same patient.

### Example 3—In Vivo Efficacy of CD63 Agonist in Treating Cancer

In this example, mice were inoculated subcutaneously with 4e5 MCA205 tumor cells on day 0. Tumor sizes were measured 2 to 3 times per week. FIG. 5, panel A, shows slight efficacy of the CD63 agonist when administered as a single therapeutic agent. Mice were treated on days 9, 12 and 15 with either 200 μg of hamster IgG control antibody or anti mouse CD63 i.p. (ThermoFisher Clone (MA5-24169) at 50 μg, 100 μg or 200 μg dose levels. With the single agent treatment of anti-CD63 0/5 mice reject their tumors. FIG. 5, panel B, shows CD63 treatment, when administered following anti-PD1 treatment starting at day 12, leads to enhanced clearance with 11/15 mice rejecting tumors. Administration of the anti-PD1 treatment alone led to 3/15 cures, demonstrating that the CD63 agonist potentiates the anti-PD1 treatment. No mice treated with control IgG rejected their tumors.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A method of treating a cell proliferative disorder, comprising:
administering to a subject having a cell proliferative disorder a therapeutically effective amount of a CD63 agonist,
wherein the CD63 agonist is administered to the subject to enhance a T cell response to abnormally proliferating cells of the cell proliferative disorder.

2. The method according to claim 1, wherein the CD63 agonist is administered to the subject independent of the level of expression of CD63 on the abnormally proliferating cells of the cell proliferative disorder.

3. The method according to claim 1 or claim 2, wherein the subject to whom the CD63 agonist is administered is receiving a T cell activation therapy.

4. The method according to claim 3, wherein the T cell activation therapy is an immune checkpoint inhibitor therapy.

5. The method according to claim 3 or claim 4, wherein the CD63 agonist is administered to the subject to potentiate the T cell activation therapy.

6. The method according to any one of claims 1 to 5, further comprising administering to the subject a T cell activator.

7. A method of treating a cell proliferative disorder, comprising:
administering to a subject having a cell proliferative disorder:
a therapeutically effective amount of a CD63 agonist; and
a therapeutically effective amount of a T cell activator.

8. The method according to claim 7, wherein the CD63 agonist is administered to the subject to enhance a T cell response to abnormally proliferating cells of the cell proliferative disorder.

9. The method according to any one of claims 1 to 8, wherein the CD63 agonist is a small molecule.

10. The method according to any one of claims 1 to 8, wherein the CD63 agonist is a peptide or polypeptide.

11. The method according to claim 10, wherein the CD63 agonist is a CD63 ligand.

12. The method according to claim 10, wherein the CD63 agonist is an antibody that specifically binds CD63.

13. The method according to claim 12, wherein the antibody that specifically binds CD63 is selected from the group consisting of: an IgG, Fv, scFv, Fab, F(ab′)₂, and Fab′.

14. The method according to any one of claims 6 to 13, wherein the T cell activator is an immune checkpoint inhibitor.

15. The method according to claim 14, wherein the immune checkpoint inhibitor is selected from the group consisting of: a cytotoxic T-lymphocyte-associated antigen 4 (CTLA-4) inhibitor, a programmed cell death-1 (PD-1) inhibitor, a programmed cell death ligand-1 (PD-L1) inhibitor, a lymphocyte activation gene-3 (LAG-3) inhibitor, a T-cell immunoglobulin domain and mucin domain 3 (TIM-3) inhibitor, an indoleamine (2,3)-dioxygenase (IDO) inhibitor, a T cell immunoreceptor with Ig and ITIM domains (TIGIT) inhibitor, a V-domain Ig suppressor of T cell activation (VISTA) inhibitor, a B7-H3 inhibitor, and any combination thereof.

16. The method according to any one of claims 6 to 13, wherein the T cell activator is an agonist of a T cell co-stimulatory receptor.

17. The method according to claim 16, wherein the T cell activator is selected from the group consisting of: an OX40 agonist, a glucocorticoid-induced TNFR-related protein (GITR) agonist, a CD137 agonist, and a CD40 agonist.

18. The method according to any one of claims 6 to 13, wherein the T cell activator is an antagonist of a T cell inhibitory signal.

19. The method according to any one of claims 6 to 13, wherein the T cell activator is a cytokine.

20. The method according to any one of claims 6 to 13, wherein the T cell activator is an agent that blocks immune suppressive cytokines.

21. The method according to claim 20, wherein the agent that blocks immune suppressive cytokines is a TGF-β receptor inhibitor.

22. The method according to any one of claims 6 to 13, wherein the T cell activator is an antagonist of an inhibitory immune receptor.

23. The method according to any one of claims 6 to 22, wherein the CD63 agonist and the T cell activator are administered concurrently.

24. The method according to any one of claims 6 to 22, wherein the CD63 agonist and the T cell activator are administered sequentially.

25. The method according to claim 24, wherein the T cell activator is administered prior to the CD63 agonist.

26. The method according to any one of claims 1 to 25, wherein the cell proliferative disorder is cancer.

27. A pharmaceutical composition, comprising:
a CD63 agonist;
a T cell activator; and
a pharmaceutically acceptable excipient.

28. The pharmaceutical composition of claim 27, wherein the CD63 agonist is a small molecule.

29. The pharmaceutical composition of claim 27, wherein the CD63 agonist is a peptide or polypeptide.

30. The pharmaceutical composition of claim 29, wherein the CD63 agonist is a CD63 ligand.

31. The pharmaceutical composition of claim 29, wherein the CD63 agonist is an antibody that specifically binds CD63.

32. The pharmaceutical composition of 31, wherein the antibody that specifically binds CD63 is selected from the group consisting of: an IgG, Fv, scFv, Fab, F(ab′)2, and Fab′.

33. The pharmaceutical composition of any one of claims 27 to 32, wherein the T cell activator is an immune checkpoint inhibitor.

34. The pharmaceutical composition of claim 33, wherein the immune checkpoint inhibitor is selected from the group consisting of: a cytotoxic T-lymphocyte-associated antigen 4 (CTLA-4) inhibitor, a programmed cell death-1 (PD-1) inhibitor, a programmed cell death ligand-1 (PD-L1) inhibitor, a lymphocyte activation gene-3 (LAG-3) inhibitor, a T-cell immunoglobulin domain and mucin domain 3 (TIM-3) inhibitor, an indoleamine (2,3)-dioxygenase (IDO) inhibitor, a T cell immunoreceptor with Ig and ITIM domains (TIGIT) inhibitor, a V-domain Ig suppressor of T cell activation (VISTA) inhibitor, a B7-H3 inhibitor, and any combination thereof.

35. The pharmaceutical composition of any one of claims 27 to 32, wherein the T cell activator is an agonist of a T cell co-stimulatory receptor.

36. The pharmaceutical composition of claim 35, wherein the T cell activator is selected from the group consisting of: an OX40 agonist, a glucocorticoid-induced TNFR-related protein (GITR) agonist, a CD137 agonist, and a CD40 agonist.

37. The pharmaceutical composition of any one of claims 27 to 32, wherein the T cell activator is an antagonist of a T cell inhibitory signal.

38. The pharmaceutical composition of any one of claims 27 to 32, wherein the T cell activator is a cytokine.

39. The pharmaceutical composition of any one of claims 27 to 32, wherein the T cell activator is an agent that blocks immune suppressive cytokines.

40. The pharmaceutical composition of claim 39, wherein the agent that blocks immune suppressive cytokines is a TGF-β receptor inhibitor.

41. The pharmaceutical composition of any one of claims 27 to 32, wherein the T cell activator is an antagonist of an inhibitory immune receptor.

42. A kit, comprising:
a pharmaceutical composition comprising a CD63 agonist; and
instructions for administering the pharmaceutical composition to a subject having a cell proliferative disorder.

43. The kit of claim 42, comprising instructions for administering the pharmaceutical composition to a subject receiving a T cell activation therapy.

44. The kit of claim 43, comprising instructions for administering the CD63 agonist to the subject to potentiate the T cell activation therapy.

45. The kit of any one of claims 42 to 44, wherein the pharmaceutical composition further comprises a T cell activator.

46. A kit, comprising:
the pharmaceutical composition of any one of claims 27 to 41; and
instructions for administering the pharmaceutical composition to a subject having a cell proliferative disorder.

47. The kit of any one of claims 42 to 46, wherein the kit comprises the pharmaceutical composition in one or more unit dosages.

48. The kit of any one of claims 42 to 46, wherein the kit comprises the pharmaceutical composition in two or more unit dosages.

49. The kit of any one of claims 42 to 48, comprising instructions for administering the pharmaceutical composition to a subject having a cell proliferative disorder independent of the level of expression of CD63 on the abnormally proliferating cells of the cell proliferative disorder.

50. A kit, comprising:
a CD63 agonist;
a T cell activator; and
instructions for administering the CD63 agonist and T cell activator to a subject having a cell proliferative disorder.

51. The kit of claim 50, wherein the kit comprises the CD63 agonist and the T cell activator in one or more unit dosages.

52. The kit of claim 50, wherein the kit comprises the CD63 agonist and the T cell activator in two or more unit dosages.

53. The kit of any one of claims 50 to 52, wherein the CD63 agonist and T cell activator are present in separate containers.

54. The kit of any one of claims 50 to 53, wherein the instructions are for administering the CD63 agonist and the T cell activator to a subject having a cell proliferative disorder independent of the level of expression of CD63 on the abnormally proliferating cells of the cell proliferative disorder.

55. The kit of any one of claims 42 to 54, wherein the CD63 agonist is a small molecule.

56. The kit of any one of claims 42 to 54, wherein the CD63 agonist is a peptide or polypeptide.

57. The kit of claim 56, wherein the CD63 agonist is a CD63 ligand.

58. The kit of claim 56, wherein the CD63 agonist is an antibody that specifically binds CD63.

59. The kit of claim 58, wherein the antibody that specifically binds CD63 is selected from the group consisting of: an IgG, Fv, scFv, Fab, F(ab′)2, and Fab′.

60. The kit of any one of claims 45 to 59, wherein the T cell activator is an immune checkpoint inhibitor.

61. The kit of claim 60, wherein the immune checkpoint inhibitor is selected from the group consisting of: a cytotoxic T-lymphocyte-associated antigen 4 (CTLA-4) inhibitor, a programmed cell death-1 (PD-1) inhibitor, a programmed cell death ligand-1 (PD-L1) inhibitor, a lymphocyte activation gene-3 (LAG-3) inhibitor, a T-cell immunoglobulin domain and mucin domain 3 (TIM-3) inhibitor, and an indoleamine (2,3)-dioxygenase (IDO) inhibitor.

62. The kit of any one of claims 45 to 59, wherein the T cell activator is an agonist of a T cell co-stimulatory receptor.

63. The kit of claim 62, wherein the T cell activator is selected from the group consisting of: an OX40 agonist, a glucocorticoid-induced TNFR-related protein (GITR) agonist, a CD137 agonist, and a CD40 agonist.

64. The kit of any one of claims 45 to 59, wherein the T cell activator is an antagonist of a T cell inhibitory signal.

65. The kit of any one of claims 45 to 59, wherein the T cell activator is a cytokine.

66. The kit of any one of claims 45 to 59, wherein the T cell activator is an agent that blocks immune suppressive cytokines.

67. The kit of claim 66, wherein the agent that blocks immune suppressive cytokines is a TGF-β receptor inhibitor.

68. The kit of any one of claims 45 to 59, wherein the T cell activator is an antagonist of an inhibitory immune receptor.

69. The kit of any one of claims 42 to 68, wherein the cell proliferative disorder is cancer.
